(19) United States
(12) Patent Application Publication
(10) Pub. No.: US 2005/0288660 A1
(43) Pub. Date: Dec. 29, 2005

(54) Method and apparatus for vaginal protection from hot fluids during endometrial ablation treatment

(76) Inventors: Thomas P. Ryan, Flemington, NJ (US); Jessica Liberatore, Marlboro, NJ (US)

Correspondence Address: Gerald T. Bodner, Esq.;Bodner & O'Rourke, LLP, Suite 108, 425 Broadhollow Road, Melville, NY, 11747, US

(21) Appl. No.: 10/870,413

(22) Filed: Jun. 17, 2004

(51) Int. Cl. A61B 18/04

(52) U.S. Cl. 606/28

(57) ABSTRACT

A fluid collection device for collecting fluid leaking from the uterus of a patient during endometrial ablation includes a collar which is mounted on a catheter of an endometrial ablation device. The collar defines with the catheter of the ablation device an interior fluid collection area. The collar has a first open end which allows leaking fluid to pass therethrough into the collection area, and is dimensioned so as to be closely contactable with the cervix of the patient undergoing treatment. Any fluid leaking from the uterus of the patient during the ablation process will be captured by the collection area of the collar. A drain tube connected to the collar removes any fluid collected by the collar. The fluid collection device removes any fluid passing from the uterus to the cervix before it may enter the vagina of the patient to cause burns or damage to the vaginal tissue lining.

### BACKGROUND OF THE INVENTION

1. Field of the Invention

This invention relates to endometrial ablation treatment, and more particularly relates to protecting the sensitive vaginal tissue during such treatments.

2. Description of the Prior Art

Excessive menstrual bleeding, or menorrhagia, is caused by a number of medical conditions, including an imbalance of the female hormones estrogen and progesterone, fibrous growths, or fibroids, in the uterus, polyps (growths on the lining of the uterus), neoplasia and blood clotting disorders (e.g., Von Willebrand's Disease). It is a well known and medically accepted practice to treat excessive menstrual bleeding non-surgically through endometrial ablation treatment, one example of which is commonly referred to as uterine balloon therapy.

More specifically, uterine balloon therapy is a minimally invasive procedure developed to reduce excessive menstrual bleeding due to benign (i.e., non-cancerous) causes, such as those identified above. It uses heat to ablate the endometrium (i.e., the lining of the uterus). With this procedure, the soft, flexible balloon attached to a thin catheter is passed through the vagina and cervix, and then placed gently into the uterus. Fluid, such as distilled water or saline, is then inserted into the balloon so that the balloon inflates to the size and shape of the uterus. The fluid is heated and circulated in the uterus for a predetermined period of time and at a predetermined temperature so as to cause necrosis and ablation of the cells on the endometrial surface. Such a device is disclosed in U.S. Pat. No. 5,084,044, which issued to Robert Quint, the disclosure of which is incorporated herein by reference.

In another endometrial ablation method, the heated fluid contacts the endometrial layer directly. The fluid is introduced at about room temperature and is heated within the uterus by a pair of electrodes which deliver RF (radio frequency) energy to the fluid. The fluid is agitated with an impeller located within the uterus itself. Such a method and device is disclosed in U.S. Pat. No. 5,653,692, which issued to Steven P. Masterson et al., the disclosure of which is incorporated herein by reference.

There are other known endometrial ablation procedures, such as using laser devices or microwave energy, but the present invention is primarily concerned with those methods which use a fluid, with or without an inflatable balloon, to treat the endometrium.

With respect to the commonly utilized techniques involving necrotizing and ablating the endometrial tissue by the application of heat, for example, using a liquid filled expandable balloon, or directly contacting the endometrium with a hot liquid, there is a cause for concern, and risk, of inadvertently burning the sensitive tissue of the vagina should the hot fluids leak through the cervix into the vagina. Leaks have been reported with the free flow (no balloon) ablation therapies, and there is a concern using balloon therapy that the balloon may tear during insertion or treatment. Clinicians who are concerned about leakage of hot fluids through the cervix often place gauze into the vagina to collect any leaks. However, this safeguard does not protect the sensitive tissue from being burned by the heated fluid.

Also, although certain uterine balloon devices currently on the market heat the circulated temperature within the balloon to approximately 87 degrees Celsius (180 degrees Fahrenheit), improved fluid-based endometrial ablation devices are projected to use even higher fluid temperatures of between about 150 degrees Celsius and about 200 degrees Celsius. At these extremely elevated temperatures, there is an even greater chance for vaginal burn if the balloon tears or is defective and hot fluids leak through the cervix.

### OBJECTS AND SUMMARY OF THE INVENTION

It is an object of the present invention to provide a method and device for preventing burns to the vaginal tissue caused by the leakage of hot fluids during fluid endometrial ablation therapy.

It is another object of the present invention to provide a device which collects hot fluids leaking from the uterus undergoing fluid endometrial ablation treatment.

It is a further object of the present invention to provide a device which might be attached to a conventional, currently marketed, fluid endometrial ablation device.

It is still another object of the present invention to provide a device which is adjustably mounted on the catheter forming part of a fluid endometrial ablation device for collecting hot fluids leaking therefrom.

It is yet a further object of the present invention to provide a fluid endometrial ablation device which includes a sensor that detects the leakage of hot fluids therefrom.

In accordance with one form of the present invention, apparatus for vaginal protection from hot fluids during endometrial ablation treatment includes an endometrial ablation device having a fluid collection device mounted thereon. The endometrial ablation device includes a catheter having a longitudinal internal fluid passageway, a distal end and a proximate end opposite the distal end. The catheter is at least partially flexible. On the distal end of the catheter is optionally mounted an inflatable balloon. The inflatable balloon has an interior which is in fluid communication with the internal fluid passageway of the catheter. The proximate end of the catheter is connected to a source of fluid, such as heated liquid, such that the fluid under pressure is directed through the catheter internal passageway and into the balloon interior. The fluid inflates the balloon to cause the balloon to contact the endometrial layer of the uterus of a patient when the balloon and distal end of the catheter is inserted into the patient's uterus.

The fluid collection device collects any fluid leaking from either or both of the balloon and the distal end of the catheter, such as where the balloon is joined to the catheter. The fluid collection device includes a collar which is mounted on the catheter and which extends longitudinally thereon over a portion of the catheter. The collar is positioned in proximity to the distal end of the catheter. The collar defines with the catheter an interior fluid collection area.

The collar has a first open end which is in communication with the interior fluid collection area defined by the collar and catheter. The collar is also dimensioned cross-sectionally so that it may be at least partially received by, or closely engageable with, the cervix of the patient. In this way, any fluid which is leaking from either the balloon or the catheter distal end through the cervix will pass through the open end of the collar and into the interior fluid collection area.

The drain tube is connected to the collar of the fluid collection device. The drain tube has an interior passageway which is in communication with the interior fluid collection area of the collar so that any fluid received by the collar may be drained therefrom using the drain tube.

The drain tube may extend into a container or vessel for ultimately collecting the fluid leaking from the endometrial ablation device and measuring the quantity of fluid which has leaked.

By having the collar closely contacting the cervix of the patient, any fluid which is leaking from the endometrial ablation device may be collected prior to its escaping through the cervix and into the vagina of the patient. Without such a device, any fluid leaking through the cervix may burn or damage the delicate vaginal tissue.

The fluid collection device has been described previously for use with an endometrial ablation device having an inflatable balloon. However, there are currently in use endometrial ablation devices which do not employ a balloon to contain the hot fluids, and these hot fluids are provided by the catheter through its distal end directly into the uterus to directly contact and ablate the endometrium. Naturally, with such a “free flow” endometrial ablation device, there is an even greater risk of leakage to the cervix and into the vagina of the patient. The fluid collection device of the present invention may be mounted on the catheter of such a “free flow” endometrial ablation device and positioned to closely engage the patient's cervix to collect any hot fluid which leaks from the uterus and into the cervix of the patient before the fluid has a chance to enter the vaginal area and cause burns or damage to the sensitive tissue therein.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIG. 1 is a partial isometric, partial cross-sectional view of an endometrial ablation device which collects fluids leaking from the device, formed in accordance with one form of the present invention, and illustrated as being positioned in the uterus of a patient and in a partially inflated state.

FIG. 2 is a partial isometric, partial cross-sectional view of the endometrial ablation device of the present invention shown in FIG. 1, illustrated as being fully inflated.

FIG. 3 is an enlarged isometric view of a fluid collection device forming part of, and selectively mounted on, the endometrial ablation device of the present invention shown in FIG. 1.

FIG. 4 is a partially exploded, isometric view of an endometrial ablation device formed in accordance with a second form of the present invention.

FIG. 5 is a partial isometric, partial cross-sectional view of an endometrial ablation device formed in accordance with a third form of the present invention and illustrating its positioning within the uterus of a patient.

FIG. 6 is an enlarged isometric view of a fluid collection device forming part of the endometrial ablation device of the present invention shown in FIG. 5.

FIG. 7 is a partial isometric, partial cross-sectional view of an endometrial ablation device formed in accordance with a fourth form of the present invention, illustrating its positioning within the uterus of a patient.

FIG. 8 is an enlarged isometric view of a fluid collection device forming part of the endometrial ablation device of the present invention shown in FIG. 7.

FIG. 9 is a partial isometric, partial cross-sectional view of a free flow endometrial ablation device formed in accordance with another form of the present invention, and illustrated as being positioned in the uterus of a patient.

### DETAILED DESCRIPTION OF THE PREFERRED EMBODIMENTS

It should be understood at the outset that the invention is directed to not only an endometrial ablation device which collects fluid leaking from the device, but also a fluid collection device usable thereon. It should also be understood that the fluid collection device of the present invention is usable in connection with either balloon-type endometrial ablation devices, or free flow endometrial ablation devices, where no balloon is used. With both types of endometrial ablation devices, there is a possibility of leakage of the hot fluids from the device. This is, of course, especially a concern with the free flow ablation devices which use no balloon to contain the hot fluids. Although gases may be used in the ablation devices, usually a heated fluid, such as hot distilled water or saline, is used. If a tear occurs in the balloon, or if the balloon separates from the distal end of the catheter to which it is attached, there is a possibility that the hot fluids used during the ablation process will leak into the uterus of the patient, through the cervix and into the patient's vagina where it may burn or damage the sensitive tissue lining the vagina.

With free flow endometrial ablation devices, no balloon is used, and the hot fluids directly contact the endometrium (i.e., the tissue lining the uterus) to cause its ablation. Since the fluids are not contained, there has occurred in many applications using such a device leakage of the hot fluids through the cervix into the patient's vagina. Physicians conventionally pack the vagina with gauze to help absorb the leaking fluids, but this procedure does not prevent burns to the vaginal tissue.

Also, it has been desirable to use ablation fluids at much greater temperatures than presently being used, such as about 150 degrees Celsius to about 200 degrees Celsius. However, at such temperatures, severe burns and tissue damage may occur if the balloon bursts or leakage of the fluid occurs through the cervix into the patient's vagina. The fluid collection device of the present invention, for use with either the balloon type or free flow type endometrial ablation devices, can recover such hot fluids leaking through the cervix of the patient before the fluid has the chance of burning or damaging the sensitive vaginal lining.

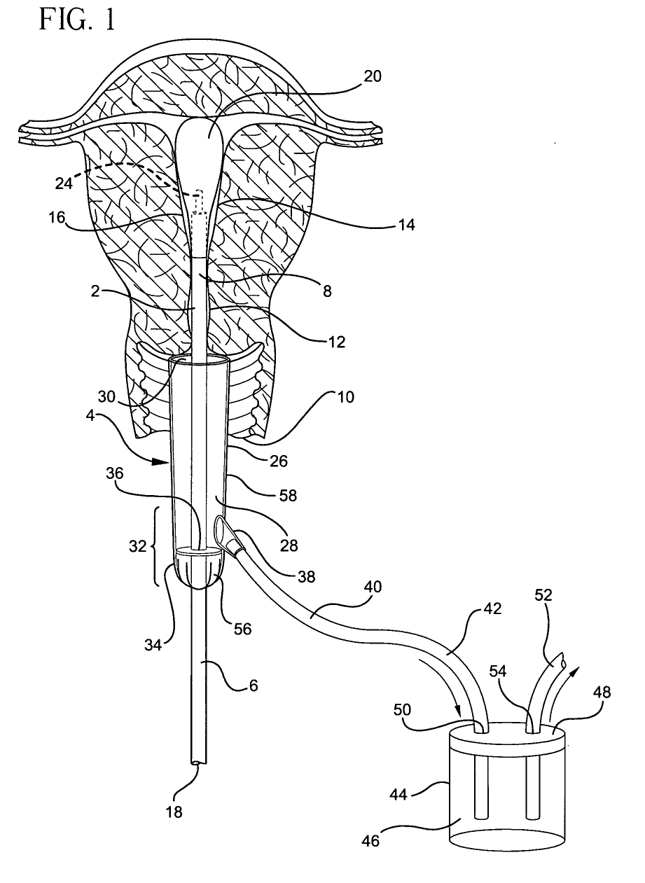

Turning initially to FIG. 1 of the drawings, an endometrial ablation device 2 having a fluid collection device 4 for collecting fluid leaking from the ablation device, formed in accordance with the present invention, is shown. Generally, the endometrial ablation device 2 includes an elongated catheter 6 which is at least partially flexible, the distal end 8 of which is manipulated by the physician or clinician through the vagina 10 and cervix 12 and into proper position in the uterus 14 of a patient to ablate the endometrium 16, i.e., the tissue lining the uterus, of the patient, in order to address the medical problems described earlier herein. The catheter has a longitudinally extending internal fluid passageway 18, a distal end 8 and a proximate end (not shown) opposite the distal end 8.

An inflatable balloon 20 is mounted on the distal end 8 of the catheter 6 of the endometrial ablation device. The balloon 20 is selected to be formed of a resilient, thermally conductive material which is biocompatible with the patient's body. The balloon thermally conducts heat from the fluid to the endometrium 16 which it contacts for a predetermined period of time to thermally ablate layers of the endometrium to a predetermined depth. The material chosen for the balloon 20 is well known in the art and is used in conventional endometrial ablation devices.

The inflatable balloon 20 defines an interior which is in fluid communication with the internal fluid passageway 18 of the catheter 6. The proximate end (not shown) of the catheter 2 is connected, and thus communicatable, with a source (not shown) of fluid 22. The fluid 22 is preferably heated distilled water or saline.

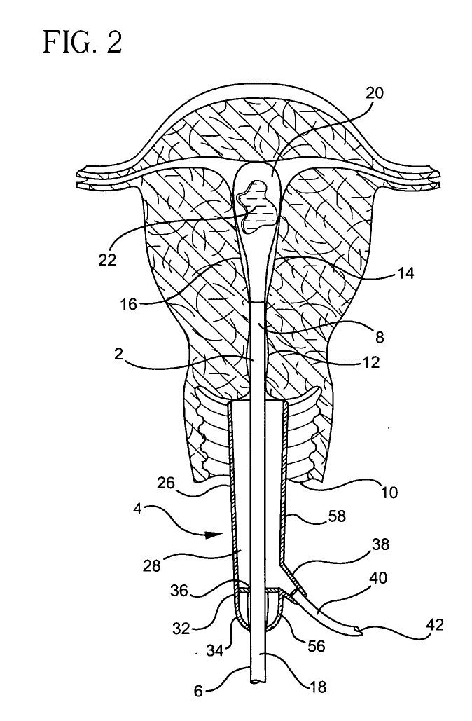

This fluid 22 is preferably directed, under pressure, through the catheter internal passageway 18 into the balloon interior such that it inflates the balloon 20, as shown in FIG. 2, to cause the balloon to contact the endometrial layer 16 of the uterus 14 of the patient undergoing treatment when, of course, the balloon 20 and distal end 8 of the catheter are properly positioned within the uterus of the patient.

The ablation device 2 also includes an internal heater 24 situated within the interior of the balloon 20 to heat the fluid 22 used in the ablation process and which fills the balloon. The heater 24 may be in the form of a coiled wire which has leads (not shown) that extend along the length of the catheter 6 and which are connected to a power source (not shown). For a more detailed description of a conventional endometrial ablation device, reference is made to U.S. Pat. No. 6,066,132 which issued to Chao Chen et al., the disclosure of which is incorporated herein by reference.

The endometrial ablation device 2 also includes a fluid collection device 4. The fluid collection device 4 is used to collect any fluids leaking from either the balloon 20 or, more generally, the distal end 8 of the catheter. The fluid collection device 4 includes a collar 26 which is generally cylindrical in shape and which is mounted on the catheter 6 and extends longitudinally on the catheter over at least a portion thereof. The collar 26 is preferably positioned in proximity to the distal end 8 of the catheter 6 so that it may contact the cervix 12 of the patient when the inflatable balloon portion of the endometrial ablation device is properly positioned within the uterus 14 of the patient.

The collar 26 defines with the catheter an interior fluid collection area 28. The collar 26 further has a first open end 30 which is in communication with the interior fluid collection area 28, and has a cross-sectional dimension so that it may be closely at least partially received within, or in contact with, the patient's cervix 12. This positioning of the collar 26, in contact with the cervix 12, allows the fluid collection device 4 to receive any fluid 22 leaking from either the balloon 20 or the catheter distal end 8, which fluids at least partially pass through the cervix 12 of the patient and into the interior fluid collection area 28 of the collar through the first open end 30 thereof.

As illustrated by FIG. 1, the collar 26 has a second end 32 which may be formed as a Y-shaped portion. The first leg 34 of the Y-shaped second end 32 has an opening 34 through which the catheter 6 passes, and the second leg 38 of the Y-shaped second end 32 is connected to a drain tube 40 to allow fluid 22 collected by the collar 26 to be drained therefrom.

More specifically, the drain tube 40 has an interior passageway 42 and is operatively coupled at its distal end to the collar 26 such that the interior passageway 42 of the drain tube is in fluid communication with the interior fluid collection area 28 of the collar 26.

The proximate end of the drain tube 40, opposite the distal end, is connected to a collection vessel 44 or reservoir. The collection vessel 44 has an interior area 46 for receiving leakage fluid 22 collected by the collar 26 and drained by the drain tube 40. Thus, the interior area 46 of the vessel is in fluid communication with the passageway 42 of the drain tube 40 for receiving fluid passing through the drain tube.

The collection vessel 44 may be sealed or sealable with a fluidtight cap 48 that has a first opening 50 through which the proximal end of the drain tube 40 passes into the interior 46 thereof. A suction tube 52 may be connected to a vacuum source (not shown), that is, a source of negative pressure, and passes through a second opening 54 formed in the cap 48 of the vessel. The vacuum source is thus operatively coupled to the drain tube 40 through the collection vessel 44 to provide suction through the drain tube and into the interior fluid collection area 28 of the collar 26 to help quickly draw any fluid 22 collected by the collar into the collection vessel 44. Suction also helps draw any fluid 22 leaking through the cervix 12 into the collar 26, and further helps to press the first end 30 of the collar against or partially into the cervix 12 to form a substantially fluidtight seal therewith.

The collar 26 may be formed from a number of various materials such as polymeric resins or thermoplastic materials, including polypropylene, polyethylene, polycarbonate, polyamide and nylon, or it may be formed from a number of metallic materials, such as stainless steel.

The fluid collection device 4 may be securely and fixedly mounted on the catheter 6 at a predetermined position thereon in relation to the distal end 8 of the catheter. Alternatively, and more desirably, the fluid collection device 4 is adjustably mounted on the catheter 6 and selectively positionable by the physician axially thereon. For this purpose, the collar 26 includes structure for adjustably positioning the collar on the catheter 6.

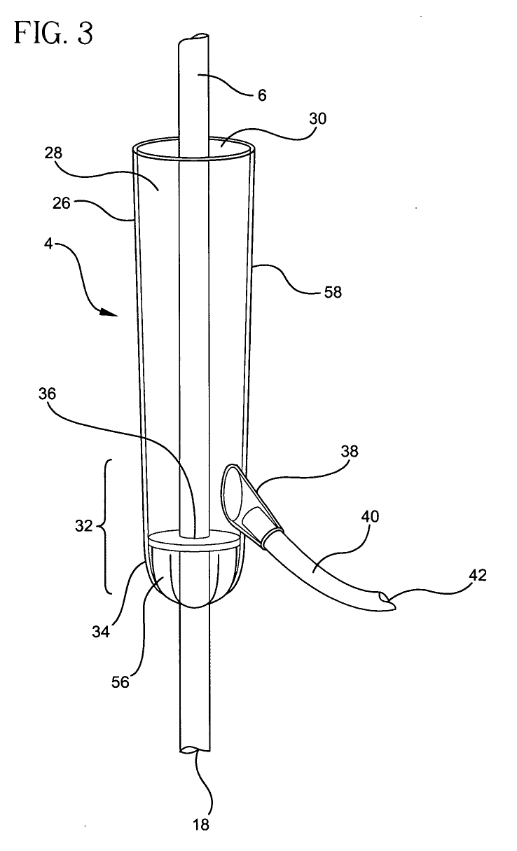

As illustrated by FIGS. 1 and 3 of the drawings, one form of such structure for adjustably positioning the collar on the catheter is where the end of the first leg of the Y-shaped second end of the collar 26 is formed with a plurality of resilient fingers 56. The resilient fingers 56 extend radially inwardly toward the catheter 6 from the main body 58 of the collar so that the free edges of the resilient fingers 56 engage the outer wall surface of the catheter 6 and exert pressure thereon. The physician may position the collar 26 by sliding it axially along the length of the catheter 6 with enough force to overcome the pressure exerted by the fingers 56 on the catheter. In the selected position, the resilient fingers 56 will maintain the collar 26 in the position selected by the physician on the catheter.

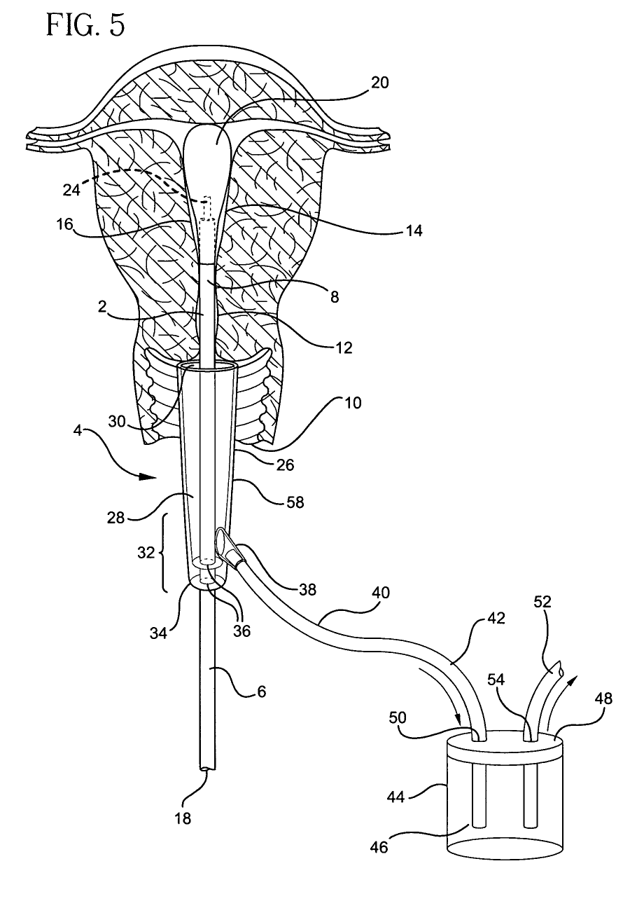

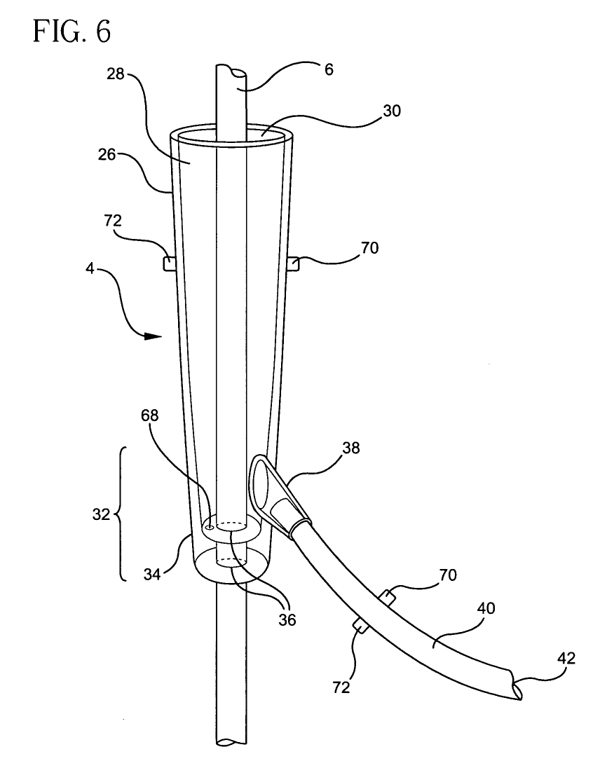

Of course, it is envisioned to be within the scope of the invention to use means other than the resilient fingers 56 described herein to adjustably position and hold the collar 26 in place on the catheter 6. A threaded compression ring (not shown) which engages a threaded end (not shown) of the first leg 34 of the collar 26 may be used, for example, where the ring may be turned in opposite directions on the threaded end of the collar 56 to loosen or tighten the engagement between the collar 26 and the catheter 6 at the opening 36 in the first leg 34 through which the catheter 6 passes. Alternatively, and as illustrated by the embodiments shown in FIGS. 5 and 6, the opening 36 in the first leg 34 of the collar through which the catheter 6 passes may be dimensioned to closely but slidingly receive the catheter, without leakage of fluid therethrough, and to exert sufficient contact force on the catheter 6 to hold the collar 26 in place during the ablation process but still allowing the physician to slide the collar along the catheter to a selected position by his overcoming this contact force.

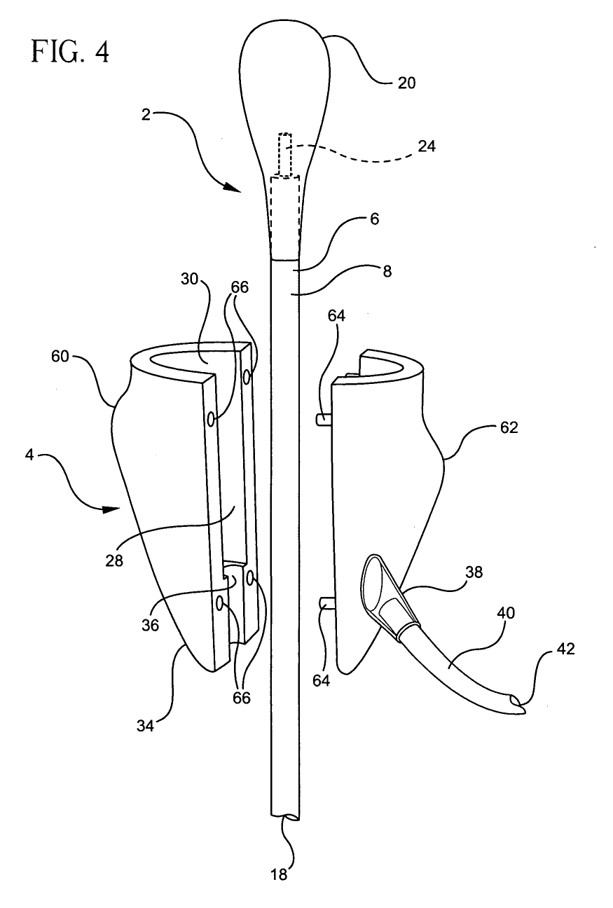

FIG. 4 illustrates another form of the present invention. As shown, the fluid collection device 4 may not be formed as part of the endometrial ablation device 2, but rather may be added to commercially available ablation devices, such as the Thermachoice™ endometrial ablation device marketed by Gynecare Worldwide, a division of Ethicon, Inc., the Caverterm™ balloon ablation device distributed by Wallsten Medical S.A. of Switzerland, the Thermablate™ balloon ablation device manufactured by MDMI Technologies Inc. of Canada, or the Hydro Thermablator™ endometrial ablation device manufactured by Boston Scientific Corporation, which is an example of a free flow endometrial ablation device. Accordingly, the collar 26 of the fluid collection device 4 may be formed as separate first and second half portions 60, 62 which are matable to define the collar 26 and the interior fluid collection area 28 of the collar. For example, as shown in FIG. 4, one half portion 62 may include pins 64 extending from an exposed surface thereof, and the other half portion 60 may include corresponding holes 66 formed in another exposed surface thereof, which holes 66 are positioned to be in alignment with the pins 64 of the corresponding mating half portion 62 so that the pins may be tightly received by the holes 66 with the catheter 6 of the ablation device placed between the two collar half portions 60, 62. To further ensure that no leakage occurs from between the mating half portions 60, 62 of the collar, one or both of the exposed surfaces of each half portion may include a seal or gasket (not shown). Each half portion 60, 62 of the collar is dimensioned in accordance with the size of the catheter 6 to which it is attached to ensure its proper closure and matability with each other and to define a fluidtight opening 36 for receiving the catheter 6 at its second end 32.

Further embodiments of the endometrial ablation device 2 and fluid collection device 4 mounted thereon and shown in FIGS. 5 and 6 are now described in greater detail. The collar 26 may be generally cone-shaped axially, with a smaller diameter at its second end 32 and a larger diameter at its first open end 30 which contacts the cervix 12 of the patient.

It may be desirable to sense if any fluid 22 is leaking from the uterus 14 to the cervix 12 and, for this purpose, the fluid collection device 4 may include a fluid detector 68. In the embodiment shown in FIG. 6, one form of a fluid detector 68 is a moisture or liquid sensor positioned within the interior fluid collection area 28 of the collar 26. In another form, the fluid detector 68 preferably includes a light source 70, such as a light emitting diode (LED), and a light receiver 72. The light source 70 and the light receiver 72 are preferably positioned diametrically opposite one another on the drain tube 40 or drain tube extension which forms part of the second leg 38 of the Y-shaped second end portion 32 of the collar 26. The light source 70 generates light which is transmitted in a light path across a portion of the passageway 42 of the drain tube and which is received by the light receiver 72. When fluid 22 is present in the passageway 42 of the drain tube, the light path will be interrupted. The light receiver 72 generates an electrical signal indicative of the fluid interrupting the light path, which signal may be applied to a monitoring device (not shown) which will set an alarm or provide some other indication to the physician that leakage of fluid has been detected. The previously described moisture sensor would similarly provide a signal indicative of the presence of fluid 22 in the interior fluid collection area 28 to such a monitoring device.

Of course, it should be realized that the light source 70 and light receiver 72 may be positioned at least partially opposite one another on the collar 26 so that the light source 70 generates light which is transmitted in a light path across a portion of the interior fluid collection area 28 (not blocked by the catheter 6 passing therethrough) of the collar 26 and which is received by the light receiver 72. The fluid 22 present in the interior fluid collection area 28 of the collar will interrupt the light path and, accordingly, the light receiver 72 will generate an electrical signal indicative of fluid interrupting the light path, which signal will be applied to a monitoring device (not shown), in the manner as described previously.

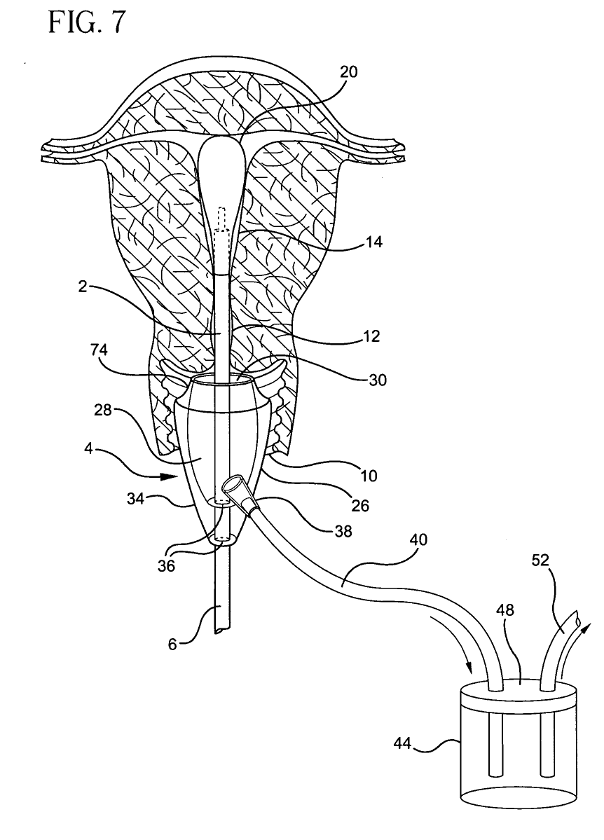

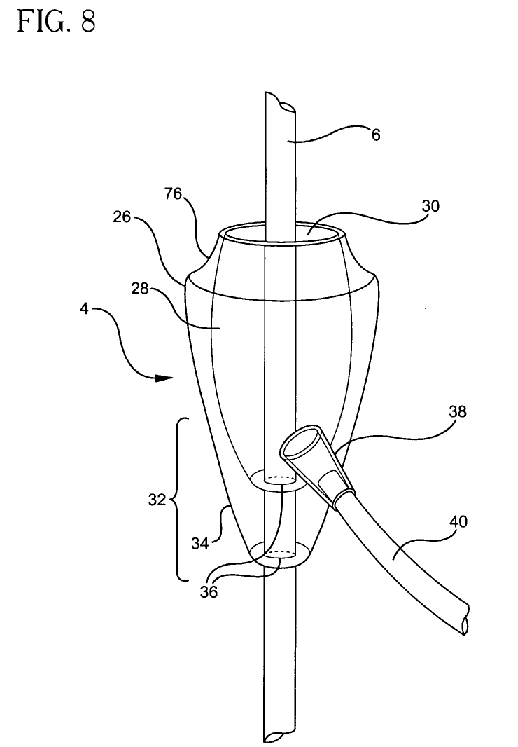

FIGS. 7 and 8 illustrate another form of the endometrial ablation device 2 and fluid collection device 4 mounted thereon and formed in accordance with the present invention. In this particular embodiment, the collar 26 is formed with a generally conically shaped first end portion that defines the first open end 30. More specifically, the first end 30 of the collar 26 recedes radially inwardly from the outermost extent of the outer surface of the collar to form a slightly concave, beveled edge 74 at the first end 30 surrounding the opening which receives leakage fluid 22 therethrough. The slightly curved edge 74 of the collar 26 is provided to closely engage the cervix 12 of a patient to form a substantially fluidtight seal therewith and to minimize any leakage through the cervix 12 past the collar 26.

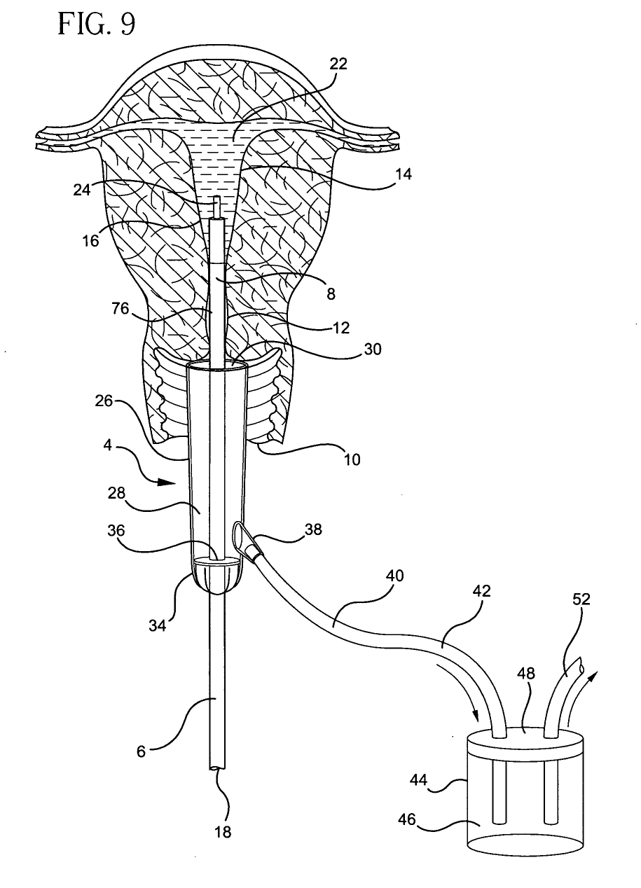

FIG. 9 illustrates a fluid collection device 4 formed in accordance with the present invention. It is in all respects similar to or the same as the fluid collection device 4 shown in FIG. 2, except that it is mounted on the catheter 6 of a free flow endometrial ablation device 76, as shown in FIG. 9, which includes no balloon nor, in this case, a heating element, and which provides heated distilled water or saline 22 directly on the endometrial layer 16 of the patient's uterus 14. The fluid collection device 4 operates to collect any fluid 22 leaking from the patient's uterus 14 and at least partially through the cervix 12 before it may enter the vaginal area 10 to cause burns or damage.

It should be realized that each of the embodiments of the present invention described herein and shown in the drawings has similar structural features and, to facilitate an understanding of the invention and its description, those common structural features have been designated with like reference numerals.

In using the endometrial ablation device 2, 76, with its fluid collection device 4 mounted thereon, the fluid collection device 4 is either pre-positioned on the catheter 6, or selectively positioned on the catheter by the physician, prior to the insertion of the ablation device transcervically into the patient. The distal end 8 of the catheter, with the attached balloon 20, if such is provided, is inserted into the uterus 14 of the patient, with the first open end 30 of the collar being received partially by, or at least contacting, the cervix 12 of the patient. Fluid 22 under pressure is then directed through the catheter 6 into the balloon 20, if such is provided, to inflate the balloon. The endometrial layer 16 of the uterus is heated to a sufficient temperature for a sufficient length of time to effectively ablate the layer. Any fluid 22 which leaks from the uterus 14 and through the cervix 12 during this process will be received by the fluid collection device 4, and in particular, the collar 26 thereof, and will be drawn through the drain tube 40 into the collection vessel 44 connected thereto, preferably by suction. The fluid 22 may be heated when it is supplied to the catheter 6 under pressure, or it may be heated by the heating element 24 situated within the balloon 20, if the ablation device includes such a heating element.

It should be understood that, although heated distilled water, saline 22, non-ionic solutions such as dextrose or glycine, and high temperature solutions, such as glycerol, may be used to ablate the endometrium 16, it is envisioned that a cryogenic cooling element mounted within the balloon 20, rather than a heating element 24, or a cooled fluid may be supplied to the endometrial ablation device in order to conduct heat from the endometrium 16 for a sufficient amount of time to effectively ablate the tissue, such as disclosed in U.S. Pat. No. 5,501,681 which issued to Robert S. Neuwirth et al., the disclosure of which is incorporated herein by reference. The cryogenic element may be connected to a conventional cryogenic refrigeration system or a conventional source of liquefied gas. It should be further realized that the term “fluid” used herein generally refers to both a liquid or a gas, as either type of fluid may be used in the endometrial ablation device of the present invention.

The fluid collection device 4 of the present invention may be built into present, commercially available ablation systems, to provide added safety for the patient. With the attachment of the collection device 4 to an endometrial ablation device and its ability to collect fluid 22 leaking from the ablation device or from the uterus 14 into the cervix 12 of the patient, higher temperatures of up to about 150 degrees Celsius to about 200 degrees Celsius may be used in the ablation devices, as the collection device will minimize any chance that a vaginal burn caused by leaking fluid at such high temperatures will occur. The fluid collection device 4 of the present invention is simple to use and fits over existing ablation devices, or it may be built in and form part of an endometrial ablation device. The fluid collection device 4 is simple in structure and low cost to manufacture, and therefore does not increase to any substantial degree the overall cost of an endometrial ablation device to which it may be attached. The fluid reservoir or vessel 44 will store any fluid which leaks from the uterus so that it may be monitored and measured. The fluid detector 68 will sound an alarm or provide an indication if fluid is detected leaking from the uterus of the patient during the ablation process.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

1. An endometrial ablation device which collects fluid leaking from the device, which comprises:
a catheter having a longitudinal internal fluid passageway, a distal end and a proximate end opposite the distal end, the catheter being at least partially flexible;
an inflatable balloon mounted on the distal end of the catheter, the inflatable balloon having an interior, the balloon interior being in fluid communication with the internal fluid passageway of the catheter, the proximate end of the catheter being communicatable with a fluid source such that fluid under pressure is directable through the catheter internal passageway into the balloon interior and is capable of inflating the balloon to cause the balloon to contact an endometrial layer of the uterus of a patient when the balloon and distal end of the catheter is inserted therein;
a fluid collection device for collecting fluid leaking from at least one of the balloon and distal end of the catheter, the fluid collection device including a collar mounted on the catheter and extending longitudinally thereon over at least a portion thereof and in proximity to the distal end thereof, the collar defining with the catheter an interior fluid collection area, the collar having a first open end in communication with the interior fluid collection area thereof, the collar having a cross-sectional dimension so as to be closely engageable with the cervix of the patient and to receive any fluid leaking from at least one of the balloon and the catheter distal end and at least partially passing through the cervix of the patient; and
a drain tube, the drain tube having an interior passageway and being operatively coupled to the collar such that the interior passageway of the drain tube is in fluid communication with the interior fluid collection area of the collar.

2. An endometrial ablation device as defined by claim 1, wherein the fluid collection device includes a collection vessel having an interior area, the interior area of the vessel being in fluid communication with the passageway of the drain tube for receiving fluid passing through the drain tube.

3. An endometrial ablation device as defined by claim 1, wherein the interior fluid collection area of the collar is selectively exposed to a negative pressure.

4. An endometrial ablation device as defined by claim 3, which further comprises a source of negative pressure, the source of negative pressure being operatively coupled to the drain tube.

5. An endometrial ablation device as defined by claim 1, wherein the fluid collection device is adjustably mounted on the catheter and selectively positionable axially thereon.

6. An endometrial ablation device as defined by claim 5, wherein the collar includes means for adjustably positioning the collar on the catheter.

7. An endometrial ablation device as defined by claim 5, wherein the collar includes a second end opposite the first open end, the second end being formed with a plurality of resilient fingers extending radially inwardly toward the catheter, the resilient fingers engaging the catheter and exerting pressure thereon to adjustably hold the collar in a selected position on the catheter.

8. An endometrial ablation device as defined by claim 1, wherein the collar includes a first portion and a second portion matable with the first portion, the first and second portions, when mated, surrounding a portion of the catheter and together defining with the catheter the interior fluid collection area of the collar.

9. An endometrial ablation device as defined by claim 1, wherein the collar includes a generally conically shaped end portion defining the first open end thereof, the conically shaped end portion being closely engageable with the cervix of a patient to form a substantially fluidtight seal therewith.

10. An endometrial ablation device as defined by claim 1, which further includes a fluid detector, the fluid detector being responsive to fluid in at least one of the interior fluid collection area of the collar and the passageway of the drain tube and providing an electrical signal indicative thereof.

11. An endometrial ablation device as defined by claim 10, wherein the fluid detector includes a light source and a light receiver, the light source and light receiver being positioned on the collar, the light source generating light which is transmitted in a light path across a portion of the interior fluid collection area of the collar and which is received by the light receiver, wherein fluid present in the interior fluid collection area of the collar will interrupt the light path, the light receiver generating the electrical signal indicative of fluid interrupting the light path.

12. An endometrial ablation device as defined by claim 10, wherein the fluid detector includes a light source and a light receiver, the light source and light receiver being positioned opposite one another on the drain tube, the light source generating light which is transmitted in a light path across a portion of the passageway of the drain tube and which is received by the light receiver, wherein fluid present in the passageway of the drain tube will interrupt the light path, the light receiver generating the electrical signal indicative of fluid interrupting the light path.

13. A fluid collection device for use with an endometrial ablation device, the endometrial ablation device including a catheter, the catheter having a longitudinal internal fluid passageway, a distal end and a proximate end opposite the distal end, the catheter being at least partially flexible, the endometrial ablation device further including an inflatable balloon mounted on the distal end of the catheter, the inflatable balloon having an interior, the balloon interior being in fluid communication with the internal fluid passageway of the catheter, the proximate end of the catheter being communicatable with a source of fluid such that fluid under pressure is directable through the catheter internal passageway into the balloon interior and is capable of inflating the balloon to cause the balloon to contact an endometrial layer of the uterus of a patient when the balloon and distal end of the catheter is inserted therein, the fluid collection device being provided to collect fluid leaking from at least one of the balloon and distal end of the catheter, the fluid collection device comprising:
a collar mounted on the catheter and extending longitudinally thereon over at least a portion thereof and in proximity to the distal end thereof, the collar defining with the catheter, when mounted thereon, an interior fluid collection area, the collar having a first open end in communication with the interior fluid collection area thereof, the collar having a cross-sectional dimension so as to be closely engageable with the cervix of the patient and to receive any fluid leaking from at least one of the balloon and the catheter distal end and at least partially passing through the cervix of the patient; and
a drain tube, the drain tube having an interior passageway and being operatively coupled to the collar such that the interior passageway of the drain tube is in fluid communication with the interior fluid collection area of the collar.

14. A fluid collection device for use with an endometrial ablation device as defined by claim 13, wherein the fluid collection device is adjustably mounted on the catheter and selectively positionable axially thereon.

15. A fluid collection device for use with an endometrial ablation device as defined by claim 14, wherein the collar includes means for adjustably positioning the collar on the catheter.

16. A fluid collection device for use with an endometrial ablation device as defined by claim 14, wherein the collar includes a second end opposite the first open end, the second end being formed with a plurality of resilient fingers extending radially inwardly toward the catheter, the resilient fingers engaging the catheter and exerting pressure thereon to adjustably hold the collar in a selected position on the catheter.

17. A fluid collection device for use with an endometrial ablation device as defined by claim 13, wherein the collar includes a first portion and a second portion matable with the first portion, the first and second portions, when mated, surrounding a portion of the catheter and together defining with the catheter the interior fluid collection area of the collar.

18. A fluid collection device for use with an endometrial ablation device as defined by claim 13, wherein the collar includes a generally conically shaped end portion defining the first open end thereof, the conically shaped end portion being closely engageable with the cervix of a patient to form a substantially fluidtight seal therewith.

19. A fluid collection device for use with an endometrial ablation device as defined by claim 13, which further comprises a fluid detector, the fluid detector being responsive to fluid in at least one of the interior fluid collection area of the collar and the passageway of the drain tube and providing an electrical signal indicative thereof.

20. A fluid collection device for use with an endometrial ablation device as defined by claim 19, wherein the fluid detector includes a light source and a light receiver, the light source and light receiver being positioned on the collar, the light source generating light which is transmitted in a light path across a portion of the interior fluid collection area of the collar and which is received by the light receiver, wherein fluid present in the interior fluid collection area of the collar will interrupt the light path, the light receiver generating the electrical signal indicative of fluid interrupting the light path.

21. A fluid collection device for use with an endometrial ablation device as defined by claim 19, wherein the fluid detector includes a light source and a light receiver, the light source and light receiver being positioned opposite one another on the drain tube, the light source generating light which is transmitted in a light path across a portion of the passageway of the drain tube and which is received by the light receiver, wherein fluid present in the passageway of the drain tube will interrupt the light path, the light receiver generating the electrical signal indicative of fluid interrupting the light path.

22. A fluid collection device for use with an endometrial ablation device, the endometrial ablation device including a catheter, the catheter having a longitudinal internal fluid passageway, a distal end and a proximate end opposite the distal end, the catheter being at least partially flexible, the distal end of the catheter being insertable into the uterus of a patient, the proximate end of the catheter being communicatable with a source of fluid such that fluid under pressure is directable through the catheter internal passageway and into the uterus to contact an endometrial layer of the uterus of a patient when the distal end of the catheter is inserted therein, the fluid collection device being provided to collect fluid leaking from the uterus and at least partially through the cervix of a patient, the fluid collection device comprising:
a collar mounted on the catheter and extending longitudinally thereon over at least a portion thereof and in proximity to the distal end thereof, the collar defining with the catheter, when mounted thereon, an interior fluid collection area, the collar having a first open end in communication with the interior fluid collection area thereof, the collar having a cross-sectional dimension so as to be closely engagable with the cervix of the patient and to receive any fluid leaking from the uterus and at least partially passing through the cervix of the patient; and
a drain tube, the drain tube having an interior passageway and being operatively coupled to the collar such that the interior passageway of the drain tube is in fluid communication with the interior fluid collection area of the collar.

23. A fluid collection device for use with an endometrial ablation device as defined by claim 22, wherein the fluid collection device is adjustably mounted on the catheter and selectively positionable axially thereon.

24. A fluid collection device for use with an endometrial ablation device as defined by claim 23, wherein the collar includes means for adjustably positioning the collar on the catheter.

25. A fluid collection device for use with an endometrial ablation device as defined by claim 23, wherein the collar includes a second end opposite the first open end, the second end being formed with a plurality of resilient fingers extending radially inwardly toward the catheter, the resilient fingers engaging the catheter and exerting pressure thereon to adjustably hold the collar in a selected position on the catheter.

26. A fluid collection device for use with an endometrial ablation device as defined by claim 22, wherein the collar includes a first portion and a second portion matable with the first portion, the first and second portions, when mated, surrounding a portion of the catheter and together defining with the catheter the interior fluid collection area of the collar.

27. A fluid collection device for use with an endometrial ablation device as defined by claim 22, wherein the collar includes a generally conically shaped end portion defining the first open end thereof, the conically shaped end portion being closely engagable with the cervix of a patient to form a substantially fluidtight seal therewith.

28. A fluid collection device for use with an endometrial ablation device as defined by claim 22, which further comprises a fluid detector, the fluid detector being responsive to fluid in at least one of the interior fluid collection area of the collar and the passageway of the drain tube and providing an electrical signal indicative thereof.

29. A fluid collection device for use with an endometrial ablation device as defined by claim 28, wherein the fluid detector includes a light source and a light receiver, the light source and light receiver being positioned on the collar, the light source generating light which is transmitted in a light path across a portion of the interior fluid collection area of the collar and which is received by the light receiver, wherein fluid present in the interior fluid collection area of the collar will interrupt the light path, the light receiver generating the electrical signal indicative of fluid interrupting the light path.

30. A fluid collection device for use with an endometrial ablation device as defined by claim 28, wherein the fluid detector includes a light source and a light receiver, the light source and light receiver being positioned opposite one another on the drain tube, the light source generating light which is transmitted in a light path across a portion of the passageway of the drain tube and which is received by the light receiver, wherein fluid present in the passageway of the drain tube will interrupt the light path, the light receiver generating the electrical signal indicative of fluid interrupting the light path.

31. A method of ablating endometrial tissue forming the interior of a uterus of a patient and minimizing burns to the tissue of the vagina of the patient due to heated fluid leaking from the uterus through the cervix of the patient during the endometrial ablation method, which comprises the steps of:
providing an endometrial ablation device which collects fluid leaking from the device, the endometrial ablation device including:
a. a catheter having a longitudinal internal fluid passageway, a distal end and a proximate end opposite the distal end, the catheter being at least partially flexible;
b. an inflatable balloon mounted on the distal end of the catheter, the inflatable balloon having an interior, the balloon interior being in fluid communication with the internal fluid passageway of the catheter, the proximate end of the catheter being communicatable with a fluid source such that fluid under pressure from the fluid source is directable through the catheter internal passageway into the balloon interior and is capable of inflating the balloon to cause the balloon to contact an endometrial layer of the uterus of a patient when the balloon and distal end of the catheter is inserted therein; and
c. a fluid collection device for collecting fluid leaking from at least one of the balloon and distal end of the catheter, the fluid collection device including a collar mounted on the catheter and extending longitudinally thereon over at least a portion thereof and in proximity to the distal end thereof, the collar defining with the catheter an interior fluid collection area, the collar having a first open end in communication with the interior fluid collection area thereof, the collar having a cross-sectional dimension so as to be closely engageable with the cervix of the patient and to receive any fluid leaking from at least one of the balloon and catheter distal end and at least partially passing through the cervix of the patient, the fluid collection device further including a drain tube, the drain tube having an interior passageway and being operatively coupled to the collar such that the interior passageway of the drain tube is in fluid communication with the interior fluid collection area of the collar;
inserting the balloon and distal end of the catheter into a uterus having an endometrial layer;
heating the endometrial layer to a sufficient temperature for a sufficient length of time to effectively ablate the layer; and
collecting any fluid leaking from at least one of the balloon and the catheter distal end of the endometrial ablation device and at least partially passing through the cervix of the patient, the fluid being receivable by the interior fluid collection area of the fluid collection device.

32. A method of ablating endometrial tissue forming the interior of a uterus of a patient and minimizing burns to the tissue of the vagina of the patient due to heated fluid leaking from the uterus through the cervix of the patient during the endometrial ablation method, which comprises the steps of:
providing an endometrial ablation device which collects fluid leaking from the device, the endometrial ablation device including:
a. a catheter having a longitudinal internal fluid passageway, a distal end and a proximate end opposite the distal end, the catheter being at least partially flexible, the distal end of the catheter being insertable into the uterus of a patient, the proximate end of the catheter being communicatable with a fluid source such that fluid under pressure from the fluid source is directable through the catheter internal passageway into the uterus to contact an endometrial layer of the uterus of a patient when the distal end of the catheter is inserted therein; and
b. a fluid collection device for collecting fluid leaking from the uterus and at least partially through the cervix of the patient, the fluid collection device including a collar mounted on the catheter and extending longitudinally thereon over at least a portion thereof and in proximity to the distal end thereof, the collar defining with the catheter an interior fluid collection area, the collar having a first open end in communication with the interior fluid collection area thereof, the collar having a cross-sectional dimension so as to be closely engagable with the cervix of the patient and to receive any fluid leaking from the uterus and at least partially passing through the cervix of the patient, the fluid collection device further including a drain tube, the drain tube having an interior passageway and being operatively coupled to the collar such that the interior passageway of the drain tube is in fluid communication with the interior fluid collection area of the collar;
inserting the distal end of the catheter into a uterus having an endometrial layer;
heating the endometrial layer to a sufficient temperature for a sufficient length of time to effectively ablate the layer; and
collecting any fluid leaking from the uterus and at least partially passing through the cervix of the patient, the fluid being receivable by the interior fluid collection area of the fluid collection device.
